(19) United States
(12) Patent Application Publication
(10) Pub. No.: US 2026/0306513 A1
(43) Pub. Date: Oct. 8, 2026

(54) PHARMACEUTICAL COMPOSITION COMPRISING ANACARDIC ACID USED FOR CELL-BASED COAGULATION

(71) Applicant: EPHYLA, ARZAL (FR)

(72) Inventor: Vincent BOURGETEAU, FEREL (FR)

(21) Appl. No.: 19/640,083

(22) Filed: Apr. 6, 2026

(30) Foreign Application Priority Data
Apr. 7, 2025 (FR) FR2503646

(51) Int. Cl. A61K 31/60; A61K 9/107; A61K 36/48

(52) U.S. Cl. CPC A61K 31/60; A61K 9/107; A61K 36/48

(57) ABSTRACT

A pharmaceutical composition includes anacardic acid for use as a cell coagulant, in particular for exfoliating the skin. The uses are aimed in particular at the coagulation of adipocytes in order to impart an anti-cellulite effect to the skin, and/or lead to the reduction of lipomas, cell-based coagulation in the cell environment inflamed around nerve cells so as to act as a painkiller agent, and at the coagulation for reducing fibrosis or a cyst.

### CROSS-REFERENCE TO RELATED APPLICATIONS

This application claims priority to French Patent Application No. 2503646, filed Apr. 7, 2025, the entire content of which is incorporated herein by reference in its entirety.

### FIELD

The present invention relates to the field of cell coagulation, also known as “dermal coagulation”, and more particularly to a pharmaceutical composition comprising anacardic acid used as cell-based coagulant or dermal coagulant, in particular for deep exfoliation of the skin.

### BACKGROUND

Skin is the main barrier of protection of the human body against external aggressions such as air pollution, climate variations, UV radiation and also against potentially harmful antigens.

The skin consists of three layers: epidermis, dermis and hypodermis.

The epidermis, which is the superficial layer in contact with the exterior, is more particularly concerned by interactions with the exterior environment. The epidermis is covered by a hydrolipidic film and is composed of four layers: stratum corneum (horny layer), stratum granulosum (granular layer), stratum spinosum (spiny layer) and stratum basale (basal layer). The epidermis normally consists of four types of cells: keratinocytes which represent 80% of all epidermis cells, the remaining 20% being composed of melanocytes, Langerhans cells or immunocompetent cells and of Merkel cells, these three groups of cells being dispersed between the keratinocytes.

The dermis is the thick layer of the skin, it is a connective tissue containing a very abundant network of blood vessels. The dermis is composed of:
protein-type macromolecules, more particularly collagen, elastin and fibronectin fibers, which form a base for the epidermis;
mucopolysaccharides which surround macromolecules; mucopolysaccharides comprise glycosaminoglycans which are proteins apt to capture water in the dermis and form a “reservoir” for hydration;
cells including fibroblasts, blood vessels consisting of endothelial cells and cells of the immune system.

The dermis plays an essential role in healing and in the elimination of toxic products.

Finally, the hypodermis, located under the dermis, is the deepest layer of the skin. The hypodermis corresponds to adipose tissue rich in adipocytes.

“Cell-based coagulation” or “cellular coagulation” refers to the solidification or semi-solidification of an organic tissue (or cell fluid); cell-based coagulation is a phenomenon that involves apoptosis or cell death. As used herein, “cell-based coagulation” is distinct from hemostatic coagulation of blood and does not refer to the activation of the coagulation cascade of blood clotting factors; rather, the term refers to the direct action of anacardic acid on the structural integrity of targeted skin cells.

### SUMMARY

Without any bound to any theory, the use according to the present invention works via an intra-cellular route, and mainly affects deviant or abnormal cells or even “cells in poor health”; in certain cases, the use can also make healthy cells coagulate.

As used throughout this specification and the appended claims, the term “cell-based coagulant” or “cellular coagulant” refers to an agent that induces the solidification, semi-solidification, or irreversible structural alteration of an organic tissue or cellular fluid through direct intracellular mechanisms, resulting in apoptosis, controlled programmed cell death, or necrosis of targeted cells, without reference to the hemostatic coagulation cascade of blood. This definition is supported by the immunofluorescence data provided herein demonstrating a significant increase in P53 expression (+7.46% in the epidermis) and a moderate increase in Caspase-3 expression (+1.86% in the epidermis; +3.90% in the dermis), both of which are markers of controlled epidermal differentiation and apoptotic processes, respectively.

As used herein, “deviant or abnormal cells” refers to cells that exhibit atypical morphology, abnormal vascularization, abnormal proliferation, or pathological changes relative to surrounding healthy tissue, including but not limited to cells constituting warts, nevi, angiomas, acrochordons, keratoses, xanthelasmas, and other benign skin lesions. As used herein, “cells in poor health” refers to cells undergoing degenerative changes, exhibiting reduced cellular function, or forming part of pathological tissue structures, including adipocytes in cellulite formations, fibrotic cells, and cells in inflamed tissue at nerve endings.

The problem at the origin of the present invention was to propose a novel agent apt to be used for cell-based coagulation leading to the exfoliation of the skin, in particular of the dermis and/or of the epidermis.

In many cases, cell-based coagulation will lead to the formation of a crust on the skin.

Among the physical methods of exfoliation of the dermis, laser-induced dermal coagulation, dermal thermal coagulation, dermal electrical coagulation or dermal cryogenic coagulation method are already known. Although effective, such methods generally lead to the destruction of cells and are often painful and/or stressful for the user. Furthermore, as already mentioned, the use according to the present invention is mainly aimed at deviant or abnormal cells or else at “cells in poor health”.

Chemical exfoliation of the dermis is mainly carried out with phenol-based compositions. Nevertheless, such solutions have drawbacks, more particularly in terms of skin sensitization.

For exfoliating the deep dermis or even the hypodermis, there is still a need for a softer, less invasive and less stressful new solution that mainly targets dying or defective cells.

Unexpectedly and beneficially, the inventor showed that the problem of cell-based coagulation could be solved using anacardic acid.

A first aspect of the invention is aimed at a pharmaceutical composition comprising anacardic acid used as an agent for cell-based coagulation.

In an embodiment, there is provided a pharmaceutical composition comprising a cell-based coagulant including anacardic acid.

The pharmaceutical composition according to the invention has the benefit of being apt to reach the dermis or even the hypodermis, even by superficial topical application. The ability to penetrate to the dermis and hypodermis is demonstrated in Examples 2.1-2.3 and 3.1, and is supported by the histological analysis of Example 3.1 showing progressive epidermal necrosis at 120 and 240 minutes of application, as well as by the dermatoscopic observations in the clinical examples showing deep coagulation of adipocytes and vascular structures.

According to a first aspect, the invention is aimed at said pharmaceutical composition to be used for treating adipocytes and imparting a slimming and/or anti-cellulite effect on the skin, and/or leading to the reduction of lipomas.

According to a second aspect, the invention is aimed at said pharmaceutical composition to be used in treating the inflamed cell environment around nerve cells and acting as a painkilling agent. The painkilling effect is achieved through cell necrosis of inflamed cells at nerve endings, as described in the clinical examples, and does not rely on any interaction with opioid or other pharmacological pain receptors.

According to a third aspect, the invention is aimed at said pharmaceutical composition to be used for reducing fibrosis or a cyst.

According to a fourth aspect, the invention is aimed at said pharmaceutical composition to be used for exfoliating the skin and/or surface defects or conditions, in particular lentigos, acrochordons (soft fibromas or pendular fibromas), cutaneous cysts, molluscum, keratoses and xanthelasmas.

In an embodiment, the invention is aimed at said pharmaceutical composition to be used for exfoliating the skin in at least one treatment chosen from the group formed by:
the treatment of atrophic scars, the curettage and the treatment of warts, the treatment of herpes pimples, the treatment of acrochordons (soft fibromas or pendular fibromas), the treatment of pigment spots or lentigos, the treatment of keratoses, the treatment of xanthelasmas, the treatment/curettage of wounds, the treatment of telangiectasia, the treatment of varicosities, the treatment of angiomas, the treatment of ruby spots, the treatment of superficial blood vessels of the skin.

Furthermore, the compositions according to the invention are stable, non-irritating, non-toxic and non-allergenic for the skin. As demonstrated in the clinical examples (Examples 2.1b, 2.2b, 2.3b, and 3.2b), the compositions elicited no reports of burning, allergic reaction, or systemic toxicity in any tested volunteer.

The present inventors have found that the cell-based coagulation activity of anacardic acid is attributable to each of its constituent 6-alkyl salicylic acid derivatives, i.e., 6-n-pentadecyl salicylic acid (C15:0), 6-[8(Z)-n-pentadecenyl] salicylic acid (C15:1), 6-[8(Z), 11(Z)-n-pentadecadienyl] salicylic acid (C15:2), and 6-[8(Z), 11(Z), 14(Z)-n-pentadecatrienyl] salicylic acid (C15:3), as well as to mixtures thereof. The activity is present whether anacardic acid is used as a substantially pure compound (98% purity; Example 1.2) or as part of a concentrated cashew shell extract (73% purity; Example 1.1), across a range of formulation types including oily compositions (Example 2.1a), Pickering emulsions (Examples 2.2a and 2.3a), transdermal patches (Examples 2.5 and 2.6), and injectable compositions (Examples 2.7-2.9).

Other aspects, benefits and properties of the present invention are presented in the following description and examples.

### BRIEF DESCRIPTION OF THE DRAWINGS

The accompanying drawings, which are incorporated in and form a part of this specification, illustrate embodiments of the invention as observed by dermatoscopic examination using a FIREFLY Digital DE300 dermatoscope (zoom ×60) during the clinical treatment studies described in Example 2.3b. In the drawings:

FIG. 1a is a dermatoscopic image taken at Day 0 (D0), prior to application of the pharmaceutical composition of Example 2.3a, showing a dysplastic nevus approximately 4 mm×4 mm in diameter, appearing bulged and vascularized, with marked hyperpigmentation on the left-hand and central areas.

FIG. 1b is a dermatoscopic image taken at Day 0 (D0) after rinsing and drying, showing agglutination of pigmentation on the surface of the dysplastic nevus and slight inflammation of the vascularization, with no particular sensation or pain reported by the patient.

FIG. 1c is a dermatoscopic image taken at Day 1 (D1), showing agglutination of pigmentation on the surface of the dysplastic nevus, cornification of the epidermis, and inflammation of the vascularization, with no particular sensation or pain reported.

FIG. 1d is a dermatoscopic image taken at Day 2 (D2), showing cornification and exfoliation of the epidermis, strong reduction of vascular inflammation, and probable vascular coagulation, with no particular sensation or pain reported.

FIG. 1e is a dermatoscopic image taken at Day 3 (D3), showing exfoliation of the epidermis, disappearance of vascularization consistent with probable coagulation, and itching on the border of the treated area.

FIG. 1f is a dermatoscopic image taken at Day 7 (D7), showing formation of a fine crust on the surface of the treated area with exfoliation on the border of the area, and very moderate itching reported by the patient.

FIG. 1g is a dermatoscopic image taken at Day 10 (D10), showing a dry crust on the surface of the treated area with the border completing exfoliation, and very slight itching reported by the patient.

FIG. 1h is a dermatoscopic image taken at Day 15 (D15), showing complete disappearance of the dysplastic nevus, a trace of deep healing at the central vascularization point, and no particular sensation or pain reported by the patient.

FIG. 2a is a dermatoscopic image taken at Day 0 (D0), prior to application of the pharmaceutical composition of Example 2.3a, showing a surface flat wart, oval-shaped, approximately 7 mm long and 3-4 mm wide, on the arm.

FIG. 2b is a dermatoscopic image taken at Day 0 (D0) after rinsing and drying, showing visible remains of the pharmaceutical formulation encrusted in the invaginated part of the wart, with no particular sensation or pain reported.

FIG. 2c is a dermatoscopic image taken at Day 1 (D1), showing visible remains of the formulation, a halo of redness around the treated area, and necrosis and exfoliation of the wart surface and of the epidermis, with no particular sensation or pain reported.

FIG. 2d is a dermatoscopic image taken at Day 2 (D2), showing necrosis and exfoliation of the epidermis at the halo area and exfoliation of the surface of the treated area, with slight itching reported in particular at the halo of redness.

FIG. 2e is a dermatoscopic image taken at Day 3 (D3), showing continued necrosis and exfoliation of the epidermis and of the surface of the treated area, with no particular sensation or pain reported.

FIG. 2f is a dermatoscopic image taken at Day 7 (D7), showing continued necrosis and exfoliation of the epidermis at the halo area and of the surface of the treated area, with no particular sensation or pain reported.

FIG. 2g is a dermatoscopic image taken at Day 10 (D10), showing continued exfoliation of the epidermis at the halo area and continuation of necrosis and exfoliation at the surface of the treated area, with no particular sensation or pain reported.

FIG. 2h is a dermatoscopic image taken at Day 15 (D15), showing continued exfoliation of the surface of the treated area with continued regression of the wart, and healthy skin on the border having returned to a normal state, with no particular sensation or pain reported.

FIG. 3a is a dermatoscopic image taken at Day 0 (D0), prior to application of the pharmaceutical composition of Example 2.3a, showing an acrochordon (soft fibroma or pendular fibroma) of approximately 2 mm on mole-type pigmentation in the fold of the arm.

FIG. 3b is a dermatoscopic image taken at Day 0 (D0) after rinsing and drying, showing necrosis of the end of the acrochordon as evidenced by a blackish coloration, and diffusion of pigmentation, with no specific sensation or pain reported.

FIG. 3c is a dermatoscopic image taken at Day 1 (D1), showing separation of the necrotic end of the acrochordon, enhanced diffusion of pigmentation, and inflammation of the microcirculation of the acrochordon with visible vasodilation, with no specific sensation or pain reported.

FIG. 3d is a dermatoscopic image taken at Day 3 (D3), showing drying of the acrochordon, disappearance of vascularization, confirmed diffusion of pigmentation, and necrosis of the epidermis around the acrochordon, with no specific sensation or pain reported.

FIG. 3e is a dermatoscopic image taken at Day 10 (D10), showing enhanced drying of the acrochordon, enhanced disappearance of vascularization, confirmed disappearance of pigmentation on the acrochordon with a slight halo of residual pigmentation, and necrosis of the epidermis around the acrochordon, with no specific sensation or pain reported.

FIG. 3f is a dermatoscopic image taken at Day 14 (D14), showing greatly enhanced drying and size reduction of the acrochordon, enhanced disappearance of vascularization, confirmed disappearance of pigmentation on the acrochordon with only a very slight and attenuated residual pigmentation halo, and no specific sensation or pain reported.

FIG. 4a is a dermatoscopic image taken at Day 0 (D0), prior to application of the pharmaceutical composition of Example 2.3a, showing a ruby angioma that is bulged, vascularized, and highly colored.

FIG. 4b is a dermatoscopic image taken at Day 0 (D0) after rinsing and drying, showing noticeable diffusion and disappearance of the color of the ruby angioma and inflammation of the vascularization, with no specific sensation or pain reported.

FIG. 4c is a dermatoscopic image taken at Day 1 (D1), showing enhanced diffusion and disappearance of the color of the ruby angioma, enhanced inflammation of the vascularization, and onset of necrosis of the vascularization, with no specific sensation or pain reported.

FIG. 4d is a dermatoscopic image taken at Day 2 (D2), showing enhanced diffusion and disappearance of the color of the ruby angioma and enhanced necrosis of the vascularization, with no specific sensation or pain reported.

FIG. 4e is a dermatoscopic image taken at Day 3 (D3), showing enhanced diffusion and disappearance of the color of the ruby angioma, enhanced necrosis of the vascularization, and onset of necrosis of the tissue surrounding the vascularization within the angioma, with no specific sensation or pain reported.

FIG. 4f is a dermatoscopic image taken at Day 7 (D7), showing enhanced diffusion and disappearance of the color of the ruby angioma and confirmed necrosis of the tissue surrounding the vascularization within the angioma, evidenced by crust formation, with no specific sensation or pain reported.

FIG. 4g is a dermatoscopic image taken at Day 10 (D10), showing near-complete disappearance of the color of the ruby angioma, absence of bulging of the skin, necrosis of the epidermis by diffusion effect around the treated area, and complete disappearance of the ruby angioma, with no specific sensation or pain reported.

### DETAILED DESCRIPTION

### Definitions

In the present text, unless otherwise specifically indicated, the percentages are expressed by weight of a reference composition.

In the present text, intervals are defined abbreviated in order not to need to describe each and all values in the interval. However, any appropriate value in the interval can be chosen as the upper value, lower value, or the end values of the interval. For example, a range of 0.1 to 1.0 represents the end values of 0.1 and 1.0, and the intermediate values of 0.2; 0.3; 0.4; 0.5; 0.6; 0.7; 0.8; 0.9 and all the intermediate intervals between 0.1 to 1.0, such as 0.2 to 0.5; 0.2 to 0.8; 0.7 to 1.0, etc. Unless otherwise specified, an interval defined as “between the value A and the value B” includes the values A and B and is thus equivalent to an interval “between the value A and the value B”, the expression “at least” comprises the value stated after, e.g. “at least 5%” should be understood as also comprising “5%”, the expression “a maximum of” comprises the value stated after, e.g. “a maximum of 5%” should be understood as also comprising “5%”.

Furthermore, in the present text, measurable values, such as a quantity, should be understood as comprising the standard deviations that can be readily determined by a person skilled in the art, in the reference art. In an embodiment, such values are intended to comprise variations of +5%.

The term “skin” refers to the epidermis, dermis, and hypodermis of the face, body, or scalp.

As used herein and in the appended claims, the term “cell-based coagulant” or “cellular coagulant” means an agent that is capable of inducing the solidification, semi-solidification, or irreversible structural alteration of an organic tissue or cellular fluid through direct intracellular mechanisms, resulting in apoptosis, controlled programmed cell death, or necrosis of one or more targeted cells. The term “cell-based coagulant” as used herein is expressly defined by the inventors and does not refer to agents that activate the hemostatic blood coagulation cascade. Support for this definition is found in: (i) the histological analysis (Example 3.1) demonstrating progressive nuclear alterations and epidermal necrosis as a function of exposure time; and (ii) the immunofluorescence analyses (Example 3.2) demonstrating a statistically significant increase in P53 epidermal marker (+7.46%) and a measurable increase in the apoptosis marker Caspase-3 (epidermis: +1.86%; dermis: +3.90%) following treatment with the composition.

As used herein, “cell-based coagulation” or “cellular coagulation” refers to the process by which a cell-based coagulant, as defined herein, induces solidification, semi-solidification, or irreversible structural alteration of organic tissue or cellular fluid through intracellular mechanisms.

As used herein, “treating” or “treatment” refers to the application of the pharmaceutical compositions of the present invention to a subject in order to reduce, eliminate, or ameliorate a skin condition or lesion described herein, including but not limited to achieving cell-based coagulation, exfoliation, necrosis, or reduction in size of the targeted tissue. The term includes prophylactic and therapeutic applications.

As used herein, “pharmaceutical composition” refers to a composition comprising anacardic acid, or a derivative or extract thereof, formulated for administration to a subject for the purpose of cell-based coagulation, skin exfoliation, or another therapeutic use described herein. The pharmaceutical composition may be formulated for topical, transdermal, or injectable administration. As used herein, the composition may also be described as a “dermatological composition” or a “topical composition” depending on the route of administration.

As used herein, “exfoliation” refers to the controlled removal of one or more layers of the skin, including the stratum corneum, the epidermis, or the dermis, through the mechanism of cell-based coagulation, necrosis, and subsequent crust formation and detachment. “Deep exfoliation” refers to exfoliation that extends at least to the dermis, and optionally to the hypodermis.

As used herein, “deviant or abnormal cells” refers to cells that exhibit atypical morphology, abnormal vascularization, abnormal proliferation, or pathological changes relative to surrounding healthy tissue, including but not limited to cells constituting warts (verrucae), nevi (including dysplastic nevi), angiomas, acrochordons, keratoses, xanthelasmas, and other benign skin lesions. “Cells in poor health” refers to cells undergoing degenerative changes, exhibiting reduced cellular function, or forming part of pathological tissue structures, including adipocytes in cellulite formations, fibrotic tissue cells, cyst-lining cells, and cells in inflamed tissue at nerve endings. These definitions are supported throughout by the clinical examples (Examples 2.1b-2.3b and 3.2b).

### Anacardic Acid

Anacardic acid is a mixture of a plurality of molecules with the following structure: a ring corresponding to salicylic acid comprising a radical —CH₂—R, the radical R being a C14 hydrocarbon radical comprising from 0 to 3 degrees of unsaturation. In particular, anacardic acid comprises the following four derivatives of 6-alkyl salicylic acid of formula I:

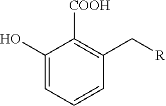

| | |
|---|---|
| number of C, | Name |
| number of | |
| [degrees of] | |
| unsaturation | |
| (on CH2R | |
| radical) | |
| C15, 0 | 6-n-pentadecyl salicylic acid |
| C15, 1 | 6-[8(Z)-n-pentadecenyl] salicylic acid |
| C15, 2 | 6-[8(Z),11(Z)-n-pentadecadienyl] salicylic acid |
| C15, 3 | 6-[8(Z),11(Z),14(Z)-n-pentadecatrienyl] salicylic |
| | acid |

More particularly, anacardic acid can be an extract of shells of cashew nuts.

According to a particular embodiment, the anacardic acid used in the present invention is chosen from 6-n-pentadecyl salicylic acid, 6-[8(Z)-n-pentadecenyl] salicylic acid, 6-[8(Z), 11(Z)-n-pentadecadienyl] salicylic acid, 6-[8(Z), 11(Z), 14(Z)-n-pentadecatrienyl] salicylic acid as well as mixtures thereof in all proportions, in particular the anacardic acid used in the present invention is chosen from 6-n-pentadecyl salicylic acid, 6-[8(Z)-n-pentadecenyl] salicylic acid, as well as mixtures thereof in all proportions.

Each of the four 6-alkyl salicylic acid derivatives of anacardic acid described herein has been demonstrated or is reasonably expected to exhibit cell-based coagulation activity based on the shared salicylic acid pharmacophore and lipophilic alkyl side chain that facilitates penetration into skin layers. The C15:0 derivative (6-n-pentadecyl salicylic acid) is specifically shown in Example 2.4 and Examples 2.9 to exhibit equivalent clinical effects to the C15:1 isomer-enriched cashew shell extracts, including superior painkilling performance.

### Extract of Shells of Cashew Nuts

According to another particular embodiment, the composition according to the present invention comprises an extract of shells of cashew nuts or western Anacardium L., said extract containing anacardic acid.

The extract of shells of cashew nuts which can be used according to the present invention can be obtained by a method of preparation comprising at least the following steps, in the following order:
providing shells of cashew nuts,
grinding the shells of cashew nuts to an average particle size of 5 mm or less,
extracting from shells of cashew nuts using ethanol at 96° at a temperature between 20° C. and 45° C.,
filtering,
recovering the filtrate,
evaporating the ethanol,
recovering the extract obtained.

More particularly, the extract of shells of cashew nuts comprises from 60 to 95% by weight of anacardic acid relative to the total weight of the extract.

Said extract is thus a concentrated extract of anacardic acid.

Furthermore, the extract of shells of cashew nuts comprises 50 to 90% by weight of 6-[8(Z)-n-pentadecenyl] salicylic acid relative to the total weight of the extract.

The concentrated extract of 6-[8(Z)-n-pentadecenyl] salicylic acid can then be distilled under vacuum to obtain 98% 6-[8(Z)-n-pentadecenyl] salicylic acid.

### Composition

The pharmaceutical composition used according to the invention may comprise from 0.1% to 100% by weight of anacardic acid, with respect to the total weight of the composition. When the anacardic acid concentration is 100% by weight, the composition consists essentially of anacardic acid, as demonstrated in Example 3.2 where the pure extract (98% 6-[8(Z)-n-pentadecenyl] salicylic acid) is applied directly without additional excipients, and effective cell-based coagulation is observed. Concentrations as low as 0.1% by weight may be used, for example, for milder or prophylactic applications. Exemplary concentrations used in the working examples range from 15% (Example 2.4) to 35% (Examples 2.3a, 2.7, 2.8, 2.9), with 25-30% being preferred in patch and Pickering emulsion formulations.

According to a first variant, the composition used according to the invention is an essentially oily composition. As used herein, the term “essentially oily composition” refers to a composition in which the continuous phase is an oily or lipophilic phase, wherein the oily phase constitutes at least 50% by weight, for example at least 80% by weight, of the total weight of the composition, and wherein the composition contains no added aqueous phase. An essentially oily composition may contain minor amounts of non-oily excipients, such as clays or organically modified phyllosilicates used as gelling or stabilizing agents, provided that such excipients do not form a continuous aqueous phase.

According to the present first variant, said composition used contains from 0.1 to 99.9% by weight, relative to the total weight of the composition, of an oily phase comprising at least one oil chosen from vegetable oils, squalane, squalene and esters of vegetable fatty substances or obtained by biotechnology.

According to an embodiment, the vegetable oily phase comprises at least one vegetable oil chosen from the group formed by sunflower oil, rapeseed oil, olive oil, camelina oil, peanut oil, coconut oil, grape seed oil, castor oil, argan oil, Djansang oil, desert date oil, hemp oil, Nigella oil, Barbary fig oil, macadamia oil, soybean oil, palm and palm tree oil, Tamanu oil, Kukui nut oil (or candle nut), sesame oil, linseed oil, walnut oil, hazelnut oil, almond oil, baobab oil, passion fruit oil, Brazil nut oil, hibiscus oil, pumpkin seed oil, Luffa oil, Carapa oil, evening primrose oil, borage seed oil, avocado oil, sea buckthorn oil, apricot kernel oil, cherry kernel oil, apple seed oil, pomegranate oil, jojoba oil, rosehip seed oil, plum kernel oil, shea butter, cocoa butter, Kokum butter, mango butter, Moabi butter, Karanja butter, Tucuma butter, Cupuaçu butter, Buriti butter, Murumuru butter, Kombo butter, Kpangnan butter and triglycerides, in particular of caprylic/capric acids.

According to a second particular variant, the composition according to the invention comprises an O/W Pickering emulsion containing an aqueous phase, a vegetable oily phase and an organically modified phyllosilicate.

Pickering emulsions are emulsions stabilized by solid particles. During the preparation of the emulsion, said solid particles are positioned at the interface between the aqueous phase and the oily phase.

Pickering emulsions used according to the present invention are oil-in-water emulsions, i.e. O/W emulsions, stabilized by a particular clay which is a modified natural phyllosilicate.

Beneficially, the aqueous phase of the Pickering emulsion is present in an amount ranging from 43% to 93%, such as 55% to 70% and for example 58% to 65% by weight relative to the total weight of the composition.

According to an embodiment, the modified natural phyllosilicate comprises a phyllosilicate chosen from the group consisting of vermiculites and smectites. In an embodiment, the phyllosilicate may be chosen from the group consisting of montmorillonites, bentonites, nonytronites, beidellites, volkonskoites, hectorites, saponites, sauconites, sobockites, stevensites, svinfordites. In an embodiment, the phyllosilicates are sodium, potassium or calcium phyllosilicates, or mixtures thereof. In an embodiment, the phyllosilicate is chosen from the group consisting of hectorite, montmorillonite, bentonite or mixtures thereof.

In an embodiment, the modified natural phyllosilicate comprises an organic compound selected from the group consisting of Xanthan gum, guar gum, Tara gum, locust bean gum, acacia gum, carrageenan, alginate, chitosan, pectin, citric acid, tartaric acid, oxalic acid, succinic acid, malic acid, acetic acid, lactic acid, propionic acid, salicylic acid, glycosaminoglycans. Beneficially, the modified natural phyllosilicate comprises a bentonite modified by Xanthan gum and by citric acid.

According to another embodiment, the modified natural phyllosilicate comprises an organic compound chosen from any organic compound known in the art of Pickering emulsions, such as same disclosed in the French Patent No. FR 2 976 503.

Beneficially, the modified phyllosilicate is present in an amount ranging from 2% to 20%, such as 2.5% to 10% and for example 3.5% to 8% by weight relative to the total weight of the composition.

Beneficially, the oily phase of the Pickering emulsion is present in an amount ranging from 5% to 45%, such as 10% to 35% and for example 15% to 30% by weight relative to the total weight of the composition.

The method of preparation of the Pickering emulsion comprises at least the following steps in the following order:
preparing the aqueous phase;
adding the organically modified phyllosilicate to the aqueous phase, and mixing;
adding the vegetable oily phase to the mixture obtained and obtaining an O/W Pickering emulsion.

Beneficially, the composition according to an aspect of the present invention comprises from 0.001 to 20% by weight of at least one agent chosen from humectants, thickeners, texturing agents, emulsifiers, dispersing agents, foaming agents, emollients, preservatives, colorants, plant extracts, plant fibers, minerals, pH adjusting agents, active ingredients and perfumes.

According to a third particular variant, the composition according to an aspect of the invention is formulated as an injectable composition. As described in Examples 2.7-2.9, the injectable composition comprises anacardic acid dissolved or dispersed in a biocompatible oily carrier, such as ethyl linoleate, in the presence of a pharmaceutically acceptable emulsifier, for example lecithin. The injectable composition is sterilized by filtration through a sterilizing filter (e.g., 0.2 μm membrane filter) prior to use. The injectable compositions of Examples 2.7-2.9 were tested in volunteers and demonstrated efficacy in treating deep plantar warts, subcutaneous hemorrhage, and localized pain, without adverse events. A person of ordinary skill in the art of pharmaceutical formulation would be able to formulate injectable compositions within the full scope of the claims based on this disclosure

A person skilled in the art will naturally take care to choose such agents so as not to alter the properties of the composition according to the invention.

### Uses

An aspect of the invention is aimed at a pharmaceutical composition comprising anacardic acid used as cell-based coagulant.

The cell-based coagulant leads to necrosis and abrasion of the epidermis and of the dermis.

According to a first aspect, the invention is aimed at the pharmaceutical composition comprising anacardic acid used for treating adipocytes and imparting a slimming and/or anti-cellulite effect to the skin, and/or leading to the reduction of lipomas.

Indeed, the use of anacardic acid leads to the reduction and release of fats by coagulation of adipocytes and thereby to imparting a slimming and/or anti-cellulite effect, and leading to the reduction of lipomas. This mechanism is supported by the clinical observations in Example 2.1b (patient with cellulite on upper arms), demonstrating progressive disappearance of cellulite, and by the observation in Example 2.1b (patient with hypodermic fatty cyst), demonstrating progressive reduction and disappearance of the fatty cyst between D0 and D14

According to a second aspect, the invention is aimed at the pharmaceutical composition comprising anacardic acid used for treating the cellular environment in inflammation around nerve cells and acting as a painkiller agent. The painkilling effect is attributed to the necrosis of inflamed cells at nerve endings, as described in Examples 2.2a, 2.3a, 2.7, 2.8, and 2.9. In injectable Examples 2.7-2.9, localized pain was substantially reduced after 30 minutes and completely eliminated after 2 hours following injection. The painkilling effect does not operate through any blood coagulation mechanism and is consistent with the inventors' defined mechanism of “cell-based coagulation.”

According to a third aspect, the invention is aimed at the pharmaceutical composition comprising anacardic acid used for reducing fibrosis or a cyst. This effect is supported by the observation in Example 2.1b (patient with hypodermic fatty cyst) demonstrating progressive reduction and disappearance of the fatty cyst from D0 to D14, consistent with cell necrosis of the cyst structure.

According to a second embodiment, the invention is aimed at the pharmaceutical composition comprising anacardic acid used for exfoliating the skin.

As shown in the examples hereinafter, such use leads to a deep exfoliation of the skin, in particular of the dermis, leading to necrosis and abrasion of the epidermis and of the dermis.

The use also leads to the coagulation of epidermal cells, i.e. the formation of a crust.

In a particular way, the pharmaceutical composition comprising anacardic acid is used to exfoliate the skin in at least one treatment chosen from the group formed by: the treatment of atrophic scars, the curettage and the treatment of warts, the treatment of herpes pimples, the treatment of acrochordons (soft fibromas or pendular fibromas), the treatment of pigmentary or lentigo spots, the treatment of keratoses, the treatment of xanthelasmas, the treatment/curettage of wounds, the treatment of telangiectasia, the treatment of varicosities, the treatment of angiomas, the treatment of ruby spots, the treatment of superficial blood vessels of the skin, in particular of the face and of the neckline.

The following examples are aimed at illustrating the present invention but do not limit the scope of the invention.

### EXAMPLES

### Example 1—Extracts of Shells of Cashew Nuts

### 1.1 Extract Preparation

The shells of cashew nuts have been mechanically cut using a knife mill so as to obtain shell particles of 5 mm or less.

100 g of cut shells of cashew nuts were then fed into a reactor with 300 g of ethanol at 96°.

The shear was then maintained under a 4-blade Misceo stirrer for 2 hours at a temperature of about 40° C. The temperature of the mixture should not exceed 45° C., in particular to prevent the conversion of anacardic acid to cardanol.

At the end of the 2 hours, the mixture was filtered through a pleated filter.

243.55 g of filtrate were obtained, that is a yield of 60.89%. The extract obtained (filtrate) comprises a dry matter content of 11.17%, that is 27.20 g.

The filtrate was then placed in a rotary evaporator at 50° C. where the evaporation of ethanol took place under vacuum. After the evaporation of ethanol (206.0 g), 4.6 g of concentrated cashew shell extract was obtained.

### Determination of the Composition of the Concentrated Extract

By gas chromatography analysis, it was possible to show that the extract obtained comprises 73% of 6-[8(Z)-n-pentadecenyl] salicylic acid, corresponding to formula I with CH2R equal to C15:1. Hereinafter in the text, the 73% extract is called “concentrated extract”.

### 1.2 Preparing the Pure Extract

In order to obtain 98% 6-[8(Z)-n-pentadecenyl] salicylic acid, the 73% concentrated extract of 6-[8(Z)-n-pentadecenyl] salicylic acid obtained hereinabove was distilled by molecular distillation under vacuum. Hereinafter in the text, the 98% extract is called “pure extract”.

### Example 2—Pharmaceutical Compositions

### Example 2.1 a Topical Composition

Table 1 shows a lipophilic topical formulation according to the invention.

TABLE 1

| Phase | Commercial name | % | INCI |
|---|---|---|---|
| A | Refined sunflower oil | 28.00 | Helianthus annuus seed oil |
| | marketed by CAUVIN | | |
| A | Myritol 318 marketed | 28.00 | Caprylic/capric triglyceride |
| | by BASF | | |
| A | OLO3D marketed | 14.00 | Stearalkonium Bentonite |
| | by Ephyla | | |
| B | Pure extract (98%) | 30.00 | Anarcardium occidentale L |
| | of shells of cashew | | (Cashew shell) extract |
| | [nuts] obtained in | | |
| | Example 1.2 | | |
| Total | | 100 | |

The constituents of phase A were mixed at 20° C. using a mechanical mixer with blades or a rotor stator tool at a speed between 3500 and 4000 rpm until homogenization, i.e. for about 7 minutes.

Phase A was then mixed with phase B at 20° C. using a mechanical mixer with blades or a rotor stator at a speed comprised between 3500 and 4000 rpm until homogenization (for about 5-7 minutes).

Such strictly lipophilic formula allows anacardic acid to penetrate deep and reach the dermis and then the hypodermis; the formula is thereby used to coagulate the cells of the deep layers of the skin; the formula has the same function as a topical patch and is applied in a substantially thick layer in order to mimic a patch.

The present composition is used for treating the deep layers of the dermis and of the hypodermis, and allows cells to coagulate to end in necrosis.

The present composition was applied on patients (volunteers) who had deep warts or fatty cysts in the hypodermis or cellulite.

3 patients were thereby treated with the composition (example 2.1 a): 1 patient with a plantar wart with a diameter of 7-8 mm; 1 patient with fatty cyst in the hypodermis of the arm, with a diameter of 6-7 mm; 1 patient with cellulite on the upper arms.

### Example 2.1 b—Treatments

### Equipment and Method

The observations were made with a dermatoscope apparatus such as the FIREFLY Digital DE300 (zoom ×60).

### Preparing the Patients

The 3 patients were prepared for treatment as follows:
1. Completing the follow-up sheet: gender, age, type of spot to be treated.
2. Putting on laboratory gloves.
3. Cleaning the area to be treated with 70% ethanol.
4. Initially observing the area with the FIREFLY Digital DE300 (zoom ×60) dermatoscope.

### Applying the Treatment

The treatment was applied as follows:
1. Centering the spot in the hole of the “corn protector” dressing and sticking the dressing to the skin.
2. Opening at the needle seal, the mini syringe vial containing the composition to be applied.
3. Placing the composition in the hole of the dressing, in direct contact with the skin.
4. Closing the seal immediately and storing the vial in a refrigerator.
5. Covering the hole of the dressing with a plaster, then fastening the whole by forming a cross with two other strips of plaster, going well over the first plaster to obtain good adhesion.
6. Application time: 6 hours.
7. Wearing gloves when removing the plasters and the dressing.
8. Preventing any spreading of the composition on the surrounding skin.
9. Rinsing the treated area with a pipette of saline or using cold clear water.
10. Gently drying with a compress.

### Care for the Treated Zone

1. Covering the area with a clean cotton dressing.
2. Changing the dressing every day for 10 to 14 days depending on local evolution

### Follow-Up and Documentation

1. Daily observation with the FIREFLY Digital DE300 (zoom ×60) dermatoscope for the first 4 days.
2. Then observation every 2-3 days until D+14.
   Observations on the Patient with Plantar Wart
1. D0: after 6 hours; the plantar wart became tinted and gained relief;
2. D1: Cornification of tissue contaminated by the plantar wart;
3. D2: deep inflammation and [inflammation] of the border of the treated area; confirmed cornification of the plantar wart;
4. D3: marked deep inflammation as well as [inflammation] of the perimeter of the treated area; confirmed cornification of the plantar wart that rises: the deep inflammation seems to push the wart upwards;
5. D5: marked deep inflammation as well as inflammation of the border of the treated area; hardening/densification of plantar wart; scalpel curettage of the wart hardened at the surface; removal of nearly 75% of the size of the wart;
6. D7: reduction of the deep inflammation and of the border of the treated area (regression of the inflammatory state), highly reduced densification of the tissue supporting the wart, and scalpel curettage of the rest of the wart; total reduction of the plantar wart;
7. D10: absence of inflammation, the skin appeared to be healthy and slightly recessed (less hypodermis and less dermis in thickness); the wart was no longer visible.

### Observations on the Patient Having a Hypodermic Fatty Cyst

1. D0: after 6 hours; the treated area was reddened by activation of blood circulation (moderate to significant hyperemia); the fatty cyst was still noticeable;
2. D1: marked hyperemia with slight swelling of the skin (deep inflammation); fatty cyst still noticeable;
3. D2: marked hyperemia, swelling of the skin (deep inflammation); the fatty cyst was still noticeable but less dense;
4. D3: ditto D2 with a slight effect of diffusion around the treated area; the fatty cyst was still noticeable but less dense;
5. D5: ditto D3 with marked hyperemia on the border of the treated area; the fatty cyst was still perceptible, but there was a real loss of dense density;
6. D7: reduction of the deep inflammation and of the border of the treated area (regression of the inflammatory state), to the touch the fatty cyst was no longer round and bulged, but soft and flattening when touched;
7 D10: slight inflammation on the treated area and the border thereof, the fatty cyst was hardly noticeable, the cyst was disappearing;
8. D14: absence of inflammation, the skin seemed healthy and was slightly recessed on the treated area (less hypodermis and less dermis in thickness); the cyst had disappeared.
   Observations on the Patient with Cellulite
1. D0: after 6 hours; the treated area was reddened by activation of blood circulation (moderate to significant hyperemia);
2. D1: marked hyperemia with slight swelling of the skin (deep inflammation);
3. D2: marked hyperemia: swelling of the skin (deep inflammation);
4. D3: ditto D2 with a slight effect of diffusion around the treated area;
5. D5: ditto D3 with marked hyperemia on the border of the treated area;
6. D7: reduction of the deep inflammation and of the border of the treated area (regression of the inflammatory state), the cellulite was no longer noticeable;
7. D10: slight inflammation on the treated area and the border thereof, the cellulite was no longer noticeable, on the contrary there was a sensation of recession: the hypodermis seemed to have refined;
8. D14: absence of inflammation, the skin seemed healthy and slightly recessed in the treated area: much less hypodermis and less dermis in thickness; the cellulite had disappeared.
The composition can also be used in treatments aimed at removing hypodermic fat, in particular fatty cysts, by necrosis, and/or treatments aimed at removing deep warts, such as plantar warts, by necrosis.

### Example 2.2 a Topical Composition

Table 2 presents a composition which can be used according to the invention containing a Pickering emulsion in which the oily phase is gelled

TABLE 2

| Phase | Commercial name | % | INCI |
|---|---|---|---|
| A | Water | Q.S. | Aqua |
| A | Frametime CX marketed | 4.50 | Bentonite & Xanthan gum & |
| | by Ephyla | | citric acid |
| A | Novasolve DMI | 3.00 | Dimethyl isosorbide |
| A | Propanediol | 6.00 | Propanediol |
| A | Sorbitol | 0.80 | Sorbitol |
| B | OLO3D marketed | 4.00 | Stearalkonium Bentonite |
| | by Ephyla | | |
| B | Pure extract (98%) | 30.00 | Anarcardium occidentale L |
| | of cashew shells | | (Cashew shell) extract |
| | obtained in | | |
| | Example 1.2 | | |
| Total | | 100 | |

On the one hand, in order to obtain a homogenized mixture, the constituents of phases A were mixed at 20° C. with a mechanical mixer with blades or a rotor stator tool at a speed comprised between 3500 and 4000 rpm, for 7 minutes. On the one hand, the constituents of phases B were mixed at 20° C. with a mechanical mixer with blades or a rotor stator tool at a speed comprised between 3500 and 4000 rpm, for 7 minutes.

Finally, in order to obtain a homogenized composition, phase B was fed into phase A and the phases were mixed with a mechanical mixer with blades or a rotor stator tool at a speed of 4000 rpm for 5-7 minutes.

The pH of the composition obtained is adjusted to 6.5-7.5.

The composition comprises a Pickering emulsion where the oily phase of phase B is gelled with the “Stearalkonium bentonite” and then encapsulated in capsules with a diameter of about ten microns. The gelation of the pure extract of shell of cashew nut followed by encapsulation in oil-in-water capsules, allows anacardic acid to be released very gradually on the surface of the skin and mainly the superficial layers of the skin, meaning the epidermis, to be treated.

The preferred method of application is to use a pierced dressing that delimits the area to be treated, the “eyelet” or the “hole” of the dressing indicates the area to be treated, then to feed the composition into the hole or the eyelet in contact with the skin and to cover the hole or the eyelet with a plaster; to leave the patch in place for a minimum of 30 minutes and up to 24 hours, then to remove the applied patch and, where appropriate, rinse the excess product from the skin with clear water.

3 patients were treated with the composition (example 2.2a); 1 patient with a 4 mm long, 1 mm diameter acrochordon on the neck, the pigmentary area at the base of the acrochordon being about 2-3 mm in diameter; 1 patient with vascularized 1-2 mm thick, 5-6 mm diameter ruby angioma on the arm; 1 patient with an irregular dysplastic nevus with very dark relief area with a diameter of 4-7 mm.

### Example 2.2 b—Treatments

### Equipment and Method

The observations were made with a dermatoscope apparatus such as the FIREFLY Digital DE300 (zoom ×60).

### Preparing the Patients

The preparation of the 3 patients to be treated was carried out in the same way as in Example 2.1 b—Treatments.

### Applying the Treatment

The treatment was applied in the same way as in Example 2.1 b—Treatments.

### Care for the Treated Zone

The treatment care was carried out in the same way as in Example 2.1 b—Treatments.

### Follow-Up and Documentation

1. Daily observation with the FIREFLY Digital DE300 (zoom ×60) dermatoscope for the first 4 days.
2. Then observation every 2-3 days until D+14.
   Observations on the Patient with an Acrochordon (Soft Fibroma or Pendular Fibroma)
1. D0: after 5 hours; the treated area turned brown by a cornification effect; the acrochordon had stiffened and appeared substantially stunted;
2. D1: the acrochordon was stiffened; the treated area was cornified;
3. D2: the acrochordon was detached; the treated area had hardened, the border seemed more vascularized;
4. D3: ditto D2 with slight hyperemizing effect of diffusion around the treated area;
5. D5: the treated area had evolved into a crust; the border was still a little red;
6. D7: the treated area had become a crust that was drying;
7. D10: the crust was dry and seemed to detach around the border thereof;
8. D14: the crust had detached; the skin was healthy without any apparent scar; the pigmentation and the acrochordon (soft fibromas or pendular fibromas) had disappeared.
   Observations on the Patient with a Vascularized Ruby Angioma,
1. D0: after 5 hours; the treated area turned brown by a cornification effect; the vascularization seemed inflamed;
2. D1: the treated area had cornified; the vascularization had coagulated and stiffened;
3. D2: ditto D1;
4. D3: ditto D2 with slight hyperemizing effect of diffusion around the treated area;
5. D5: the treated area had evolved into a crust; the border was still a little red;
6. D7: the treated area had become a crust that was drying;
7. D10: the crust was dry and seemed to detach around the border thereof;
8. D14: the crust had detached; the skin was healthy with no apparent scar; the pigmentation and the ruby angioma had disappeared.
   Observations on the Patient with Dysplastic Nevus
1. D0: after 6-7 h; the treated area turned brown by a cornification effect; the vascularization seemed inflamed on the raised area of the nevus;
2. D1: the treated area had cornified; the vascularization had coagulated and stiffened;
3. D2: ditto D1;
4. D3: ditto D2 with slight hyperemizing effect of diffusion around the treated area;
5. D5: the treated area had evolved into a crust; the border was in red hyperemia;
6. D7: the treated area had become a crust that was drying; the border was showing red hyperemia as in D5;
7. D10: the crust was dry and seemed to detach around the border thereof; the border was still showing hyperemia;
8. D14: the crust had detached; the skin was healthy without any apparent scar; the dysplastic nevus had disappeared; hyperemia on the border was still present but with low intensity.

The above was thus a coagulation leading to exfoliation; the treatment was particularly effective for removing, by necrosis and exfoliation, acrochordons (soft fibromas or pendular fibromas), scars, stretch marks, pigmentary lesions (melasma), neva, especially dysplastic neva, age spots or lentigos, unattractive moles such as ruby angiomas or dysplastic neva. The coagulation of blood micro-vessels observed in the case of ruby angiomas further sustains allows for application for stopping bleeding, especially when skin cuts occur and affect the dermis and/or hypodermis.

It should be noted that the content of the present composition penetrates through the epidermis to reach the dermis as well, which makes it possible, in other volunteers, at the dermal-epidermal junction, to inhibit pain by cell necrosis of cells inflamed at the nerve endings, said composition thus being particularly recommended for the curettage of wounds, in which case the application should not exceed 4 hours and [should last] at least 1 hour: the application as a poultice acts, without any mechanical action, as a curettage.

### Example 2.3 a Topical Composition

Table 3 presents a composition which can be used according to the invention containing a Pickering emulsion in which the oily phase is gelled

TABLE 3

| Phase | Commercial name | % | INCI |
|---|---|---|---|
| A | Water | Q.S. | Aqua |
| A | Frametime CXG | 5.0 | Bentonite & Xanthan gum |
| | marketed by Ephyla | | & sodium stearoyl glutamate |
| | | | & citric acid |
| A | Sodium benzoate | 0.40 | Sodium benzoate |
| A | Citric acid monohydrate | 0.40 | Citric acid |
| B | OLO3D marketed by | 4.50 | Stearalkonium Bentonite |
| | Ephyla | | |
| B | Concentrated extract | 35.00 | Anarcardium occidentale L |
| | (73%) of shells of | | (Cashew shell) extract |
| | cashew [nuts] obtained | | |
| | in Example 1.1 | | |
| Total | | 100 | |

On the one hand, in order to obtain a homogenized mixture, the constituents of phases A were mixed at 20° C. with a mechanical mixer with blades or a rotor stator tool at a speed comprised between 3500 and 4000 rpm, for 7 minutes. On the one hand, the constituents of phases B were mixed at 20° C. with a mechanical mixer with blades or a rotor stator tool at a speed comprised between 3500 and 4000 rpm, for 7 minutes.

Finally, in order to obtain a homogenized composition, phase B was fed into phase A and the phases were mixed with a mechanical mixer with blades or a rotor stator tool at a speed of 4000 rpm for 5-7 minutes.

The pH of the composition obtained is adjusted to 5.0-5.5.

The present composition comprises a Pickering emulsion where the oily phase (phase B) is gelled with “Stearalkonium bentonite” and then encapsulated in capsules of approximately 5 to 10 microns in diameter. The gelation of the concentrated extract of shells of cashew [nuts] followed by a capsule-with-water encapsulation, allows the anacardic acid to gradually release on the surface of the skin and to treat mainly the superficial layers of the skin, namely the epidermis, and to coagulate the cells.

The preferred method of application is to use a pierced dressing that delimits the area to be treated, where the “eyelet” or the “hole” of the dressing indicates the area to be treated, to feed the composition into the hole or the eyelet in contact with the skin and to cover the hole or the eyelet with a plaster, to leave the patch in place for a minimum of 30 minutes and up to 24 hours, then to remove the applied patch and, where appropriate, to rinse the excess product from the skin with clear water.

After an application to volunteer patients, it was observed that the epidermis coagulated and turned into a crust after 48 to 96 hours, the crust then disappeared after approximately 10 to 14 days, leaving a clean and flawless healed skin: the problem or the flaw disappeared into the crust. The above is thus an exfoliation; the treatment is particularly effective for removing, by necrosis and exfoliation, acrochordons (soft fibromas or pendular fibromas), scars, stretch marks, pigmentary lesions (melasma), neva, especially dysplastic neva, age spots or lentigos, unattractive moles such as ruby angiomas. The coagulation of blood micro-vessels observed in the case of ruby angiomas further allows for application to for stopping bleeding, especially when skin cuts occur and affect the dermis and/or hypodermis.

It has also been observed that the present composition penetrated the surface quickly which led, in other volunteers, to inhibiting pain by cell necrosis in the skin, said composition being particularly further recommended for the curettage of wounds, in which case the application should not exceed 4 hours and [should last] at least 1 hour: the application as a poultice acts, without any mechanical action, as a curettage.

Thereby, 4 patients were treated with the present composition (Example 2.3 a); 1 patient with irregular dysplastic nevus with a very dark relief area with a diameter of 4-7 mm, on the upper back; 1 patient with an oval shaped, approximately 7 mm long, 3-4 mm wide papilloma wart on the arm; 1 patient with an acrochordon (soft fibroma or pendular fibroma) on 5 mm long neva in the fold of the arm, where the spot (neva) had a diameter of 4 mm on the surface of the skin; 1 patient with a ruby angioma (vascularized).

### Example 2.3 b—Treatments

### Equipment and Method

The observations were made with a dermatoscope apparatus such as the FIREFLY Digital DE300 (zoom ×60).

### Preparing the Patients

The preparation of the 3 patients to be treated was carried out in the same way as in Example 2.1 b—Treatments.

### Applying the Treatment

The treatment was applied in the same way as in Example 2.1 b—Treatments.

### Care for the Treated Zone

The treatment care was carried out in the same way as in Example 2.1 b—Treatments.

### Follow-Up and Documentation

1. Daily observation with the FIREFLY Digital DE300 (zoom ×60) dermatoscope for the first 4 days.
2. Then observation every 2-3 days until D+14.
   Observations on the Patient with Dysplastic Nevus:

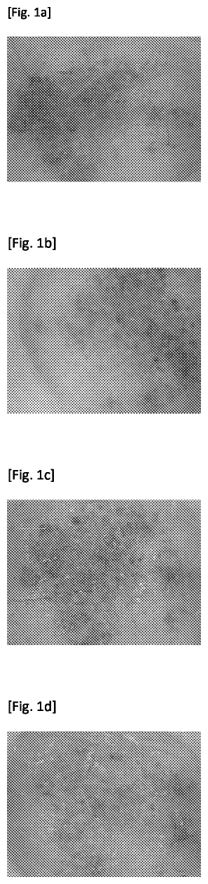

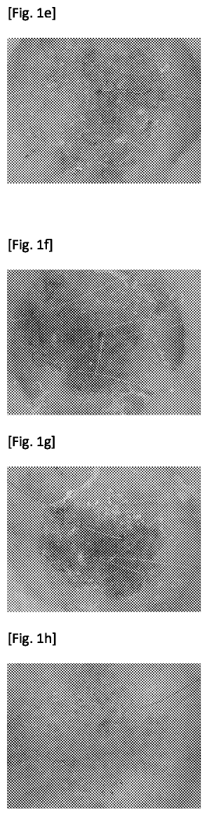

The photographs in FIGS. 1a-1h correspond to the following observations:
1. D0: before application: Dysplastic nevus 4 mm long by 4 mm wide, bulged and vascularized on the left-hand area (with marked hyperpigmentation) and the central area—FIG. 1a;
2. D0 after rinsing/drying: no special sensation/pain; agglutination of pigmentation on the surface, slight inflammation of the vascularization—FIG. 1b;
3. D1; no particular sensation/pain; agglutination of pigmentation on the surface and cornification of the epidermis, inflammation of the vascularization—FIG. 1c;
4. D2: No particular sensation/pain; cornification and exfoliation of the epidermis; strong reduction of vascular inflammation and probable vascular coagulation—FIG. 1d;
5. D3: Itching on the border of the treated area; exfoliation of the epidermis; disappearance of vascularization (probable coagulation)—FIG. 1e;
9. D7: Very moderate itching; formation of a fine crust on the surface of the treated area with exfoliation on the border of the area—FIG. 1 f;
10. D10: Very slight itching probably related to the presence of a thin skin crust on the treated area; the surface of the crust was dry, the border was ending the exfoliation thereof—FIG. 1g;
11. D15: No particular sensation/pain; dysplastic nevus disappeared; trace of deep healing at the central vascularization point—FIG. 1h.
   Observations on the Patient with a Wart:

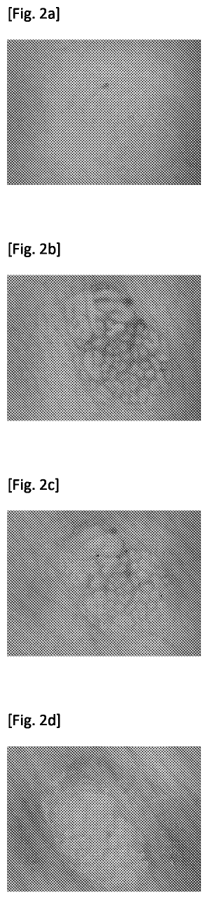

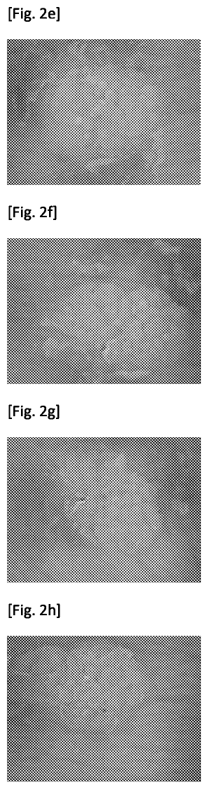

The photographs in FIGS. 2a-2h correspond to the following observations:
1. D0: before application: surface flat wart—FIG. 2a;
2. D0 after rinsing/drying: remains of the formulation were visible (encrustation of the pharmaceutical formulation in the invaginated part of the wart); no particular sensation/pain—FIG. 2b;
3. D1: Remains of the formulation were still visible (encrustation of the pharmaceutical formulation in the invaginated part of the wart); no particular sensation/pain although there was a halo of redness around the treated area; necrosis/exfoliation of the wart surface and of the epidermis—FIG. 2c;
4. D2: Slight itching in particular at the halo of redness around the treated area; necrosis & exfoliation of the epidermis at said area (halo); exfoliation of the surface of the treated area—FIG. 2d;
5. D3: No particular sensation/pain; necrosis and exfoliation of the epidermis in said area (halo); exfoliation of the surface of the treated area—FIG. 2e;
6. D7: No particular sensation/pain; necrosis & exfoliation of the epidermis in said area (halo); continued necrosis & exfoliation of the surface of the treated area—FIG. 2f;
7. D10: No particular sensation/pain; exfoliation of the epidermis in said area (halo); continuation of the necrosis & exfoliation of the surface of the treated area—FIG. 2g;
8. D15: No particular sensation/pain; exfoliation of the epidermis in said area (halo); continuation of the exfoliation of the surface of the treated area; the wart continued to react and to regress while the skin on the border seemed to have returned to the normal state thereof—FIG. 2h.
   Observations about the Patient with an Acrochordon (Soft Fibroma or Pendular Fibroma):

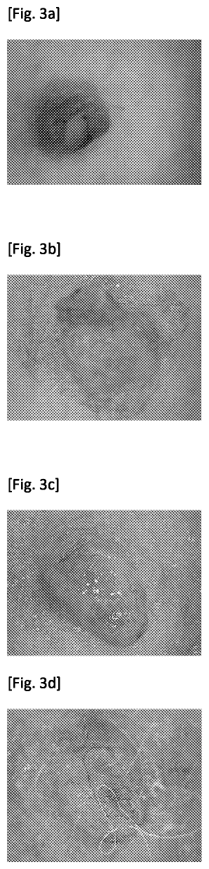

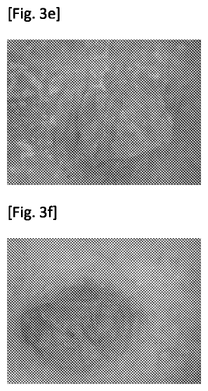

The photographs in FIGS. 3a-3f correspond to the following observations:
1. D0: before application: there is a problem with the zoom, but there was an acrochordon (soft fibroma or pendular fibroma) of about 2 mm on mole type pigmentation—FIG. 3a;
2. D0: after rinsing/drying: no specific sensation/pain, necrosis of the end of the acrochordon (blackish color), diffusion of pigmentation—FIG. 3b;
3. D1: No specific sensation/pain, the necrosis of the end of the acrochordon (blackish color) had separated; enhanced diffusion of pigmentation; inflammation of the microcirculation of the acrochordon (visible vasodilation)—FIG. 3c;
4. D3: No specific sensation/pain, drying out of the acrochordon and disappearance of vascularization; confirmed diffusion of pigmentation; necrosis of the epidermis around the acrochordon—FIG. 3d;
5. D10: No specific sensation/pain, enhanced drying out of the acrochordon and enhanced disappearance of the vascularization; confirmed disappearance of pigmentation on the acrochordon (slight halo of pigmentation around the rest of the acrochordon; necrosis of the epidermis around the acrochordon—FIG. 3e;
6. D14: No specific sensation/pain, enhanced drying out of the acrochordon (very large reduction in size) and enhanced disappearance of the vascularization; confirmed disappearance of pigmentation on the acrochordon (very slight halo of pigmentation around the rest of acrochordon, but very attenuated compared to the original pigmentation)—FIG. 3f.
   Observations on the Patient with Ruby Angioma:

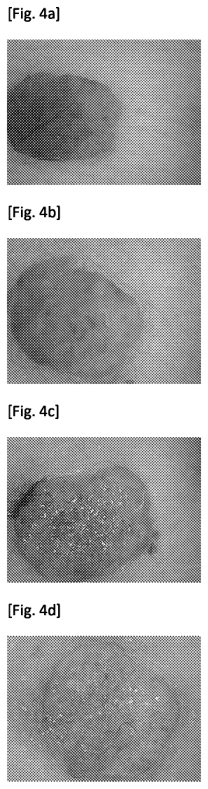

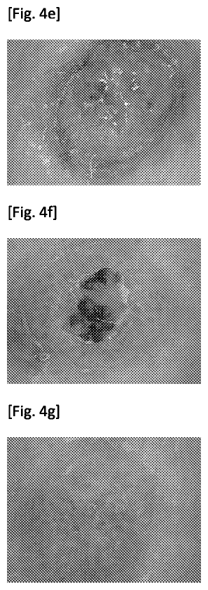

The photographs in FIGS. 4a-4g correspond to the following observations:
1. D0: before application: Bulged, vascularized and highly colored mole—FIG. 4a;
2. D0 after rinsing/drying: No specific sensation/pain, diffusion (noticeable disappearance) of the color of the mole; inflammation of the vascularization—FIG. 4b;
3. D1: No specific sensation/pain, enhanced diffusion (noticeable disappearance) of the color of the mole comforted; enhanced inflammation of the vascularization and necrosis of the vascularization—FIG. 4c;
4. D2: No specific sensation/pain, enhanced diffusion (noticeable disappearance) of the color of the mole; enhanced necrosis of the vascularization—FIG. 4d;
5. D3: No specific sensation/pain, enhanced diffusion (noticeable disappearance) of the color of the mole; enhanced necrosis of the vascularization, necrosis of the flesh around the vascularization inside the mole—FIG. 4e;
6. D7: No specific sensation/pain, enhanced diffusion (noticeable disappearance) of the color of the mole; necrosis of the flesh around the vascularization inside the mole confirmed by a crust—FIG. 4f;
7. D10 No specific sensation/pain, necrosis of the epidermis by an effect of diffusion around the treated area. The color disappeared almost entirely and the skin was no longer bulged; the ruby angioma disappeared—FIG. 4g.

### Example 2.4 Topic Composition

Table 4 shows a composition which can be used according to the invention and contains 6-n-pentadecyl salicylic acid, anacardic acid in C15:0.

TABLE 4

| Phase | Commercial name | % | INCI |
|---|---|---|---|
| A | Water | Q.S. | Aqua |
| C | Cutina GMS | 4.50 | Glyceryl monostearate |
| C | Cetearyl alcohol | 1.50 | Cetearyl alcohol |
| C | Amisoft HS-11P | 0.30 | Sodium stearoyl glutamate |
| B | Glycerin | 3.50 | glycerin |
| B | Xanthan gum FF marketed | 0.15 | Xanthan gum |
| | by Jungbunzlauer | | |
| C | Refined sunflower oil | 25.00 | Helianthus annuus seed |
| | marketed by CAUVIN | | oil |
| D | 6-n-pentadecyl | 15.00 | |
| | salicylic acid marketed | | |
| | by Sigma Aldrich Merck | | |
| E | Citric acid monohydrate | 0.40 | Citric acid |
| Total | | 100 | |

On the one hand, in order to obtain a homogenized mixture, the constituents of phases A were mixed at 20° C. with a mechanical mixer with blades or a rotor stator tool at a speed comprised between 3500 and 4000 rpm, for 7 minutes. On the one hand, the constituents of phases B were mixed at 20° C. with a mechanical mixer with blades or a rotor stator tool at a speed comprised between 3500 and 4000 rpm, for 7 minutes.

Finally, in order to obtain a homogenized composition, phase B was fed into phase A and the phases were mixed with a mechanical mixer with blades or a rotor stator tool at a speed of 4000 rpm for 5-7 minutes.

The pH of the composition obtained is set to 5.0.

The preferred method of application is to use a pierced dressing that delimits the area to be treated, where the “eyelet” or the “hole” of the dressing indicates the area to be treated, to feed the composition into the hole or the eyelet in contact with the skin and to cover the hole or the eyelet with a plaster, to leave the patch in place for a minimum of 30 minutes and up to 24 hours, then to remove the applied patch and, where appropriate, to rinse the excess product from the skin with clear water.

The present composition was applied under the same conditions as the conditions of the composition of Example 2.3 hereinabove, the effects observed following the applications are similar to the effects of the composition of Example 2.3. It was noted, however, that the composition of Example 2.4 has superior effectiveness for deep curettage on deeper wounds, with an optimization of the painkilling effect.

### Example 2.5 Composition in the Form of a Transdermal Device (Patch)

Table 5 shows a “patch” composition which can be used according to the invention.

TABLE 5

| Phase | Brand name | % | INCI |
|---|---|---|---|
| A | Myritol 318 | 70.00 | Caprylic: capric triglyceride |
| | marketed by BASF | | |
| A | Vitamin C TetraE | 5.0 | Ascorbyl tetraisopalmitate |
| | marketed by Ephyla | | |
| B | Pure extract (98%) | 25.00 | Anarcardium occidentale L |
| | of shells of cashew | | (Cashew shell) extract |
| | [nuts] obtained in | | |
| | Example 1.2 | | |
| Total | | 100 | |

The constituents of phases A and B were mixed at 20° C. under slow stirring with a single mixing blade at a speed of 300-800 rpm.

The present composition is intended to be applied as a patch over a period of 2 to 6 hours. Said composition is aimed at the deep layers of the dermis and of the hypodermis and is aimed at coagulating the cells so as to end in necrosis. The composition was applied for 2 to 6 hours depending on the person (volunteers), then rinsed with clean water. After 48 to 96 hours, the adipocytes, and more particularly the fatty cysts or the tissues contaminated by a wart disappeared via necrosis.

The composition can be used in treatments aimed at removing, via necrosis, hypodermic fat, more particularly fatty cysts, by necrosis, or else for removing warts via necrosis.

On other volunteers having skin imperfections such as small angiomas or flat or raised ruby spots or unattractive apparent blood vessels, the composition was applied for 2 to 3 hours depending on the person (volunteers), then rinsed with clear water. After 48 to 96 hours, the flaws mentioned disappeared via necrosis.

### Example 2.6 Composition in the Form of a Transdermal Device (Patch)

Table 6 shows a “patch” composition which can be used according to the invention.

TABLE 6

| Phase | Commercial name | % | INCI |
|---|---|---|---|
| A | Myritol 318 marketed | 70.00 | Caprylic/capric triglyceride |
| | by BASF | | |
| A | Vitamin C TetraE | 5.0 | Ascorbyl tetraisopalmitate |
| | marketed by Ephyla | | |
| B | Concentrated extract | 25.00 | Anarcardium occidentale L |
| | (73%) of cashew | | (Cashew shell) extract |
| | shells obtained in | | |
| | Example 1.1 | | |
| Total | | 100 | |

The constituents of phases A and B were mixed at 20° C. under slow stirring with a single mixing blade at a speed of 300-800 rpm.

The present composition can be used for treating the deep layers of the dermis and of the hypodermis, leading to the coagulation of cells which ends in necrosis. Said composition was applied for 2 to 6 hours depending on the person (volunteers), then rinsed with clear water. After 48 to 96 hours, the adipocytes, and more particularly the fatty cysts or the tissues contaminated by a wart disappeared via necrosis.

The present composition can be used in treatments aimed at removing hypodermic fat via necrosis, more particularly fatty cysts and/or in treatments aimed at removing warts via necrosis.

On other volunteers having skin imperfections such as small angiomas or flat or raised ruby spots or unattractive apparent blood vessels, the composition was applied for 2 to 3 hours depending on the person (volunteers), then rinsed with clear water. After 48 to 96 hours, the mentioned flaws disappeared via necrosis.

### Example 2.7 Injectable Composition

Table 7 shows an injectable composition according to the invention.

TABLE 7

| Phase | Brand name | % | INCI |
|---|---|---|---|
| A | EPHYSTER ECR | 60.00 | Ethyl linoleate |
| | marketed by Ephyla | | |
| A | TecFis QF marketed | 5.0 | Lecithin |
| | by Fismerlecithin | | |
| B | Concentrated extract | 35.00 | Anarcardium occidentale L |
| | (73%) of shells of | | (Cashew shell) extract |
| | cashew [nuts] obtained | | |
| | in Example 1.1 | | |
| Total | | 100 | |

The constituents of phases A and B were mixed at 20° C. under slow stirring with a single mixing blade at a speed of 300-800 rpm. Then the mixture underwent sterilizing filtration.

The present composition can be used for treating the deep layers of the dermis and of the hypodermis.

In volunteers with deep warts in the skin tissue, in particular on the sole of the foot, necrosis of the wart and of the contaminated tissue is observed approximately 96 hours after a localized injection.

In volunteers with subcutaneous hemorrhage, an injection of the composition on the area stopped, in a few minutes, the hemorrhage (cessation of enlargement related to internal bleeding) by coagulation of the injured endothelial cells; it was also observed that the hematoma was less intense and disappeared rapidly.

In volunteers with localized pain, an injection with a needle directly on the area to be treated substantially reduced pain after 30 minutes and completely reduced pain after 2 hours, with a lasting effect.

### Example 2.8 Injectable Composition

Table 8 shows an injectable composition according to the invention.

TABLE 8

| Phase | Brand name | % | INCI |
|---|---|---|---|
| A | EPHYSTER ECR | 60.00 | Ethyl linoleate |
| | marketed by Ephyla | | |
| A | TecFis QF marketed | 5.0 | Lecithin |
| | by Fismerlecithin | | |
| B | Pure extract (98%) | 35.00 | Anarcardium occidentale L |
| | of shells of cashew | | (Cashew shell) extract |
| | [nuts] obtained in | | |
| | Example 1.2 | | |
| Total | | 100 | |

The constituents of phases A and B were mixed at 20° C. under slow stirring with a single mixing blade at a speed of 300-800 rpm. Then the mixture underwent sterilizing filtration.

The present composition can be used for treating the deep layers of the dermis and of the hypodermis.

In volunteers with deep warts in the skin tissue, in particular on the sole of the foot, necrosis of the wart and of the contaminated tissue is observed approximately 96 hours after a localized injection.

In volunteers with subcutaneous hemorrhage, an injection of the composition on the area stopped, in a few minutes, the hemorrhage (cessation of enlargement related to internal bleeding) by coagulation of the injured endothelial cells; it was also observed that the hematoma was less intense and disappeared rapidly.

In volunteers with localized pain, an injection with a needle directly on the area to be treated significantly reduced pain after 30 minutes and completely reduced pain after 2 hours, with a lasting effect.

### Example 2.9 Injectable Composition

Table 9 shows an injectable composition according to the invention.

TABLE 9

| Phase | Brand name | % | INCI |
|---|---|---|---|
| A | EPHYSTER ECR | 60.00 | Ethyl linoleate |
| | marketed by Ephyla | | |
| A | TecFis QF marketed | 5.0 | Lecithin |
| | by Fismerlecithin | | |
| B | 6-n-pentadecyl | 35.00 | |
| | salicylic acid | | |
| | marketed by | | |
| | Sigma Aldrich Merck | | |
| Total | | 100 | |

The constituents of phases A and B were mixed at 20° C. under slow stirring with a single mixing blade at a speed of 300-800 rpm. Then the mixture underwent sterilizing filtration.

The present composition can be used for treating the deep layers of the dermis and of the hypodermis. In volunteers who have deep warts in the skin tissue, in particular on the sole of the foot, necrosis of the wart and of the contaminated tissue was observed approximately 96 hours after a localized injection.

In volunteers with subcutaneous hemorrhage, an injection of the composition on the area stopped, in a few minutes, the hemorrhage (cessation of enlargement related to internal bleeding) by coagulation of the injured endothelial cells; it was also observed that the hematoma was less intense and disappeared rapidly.

In volunteers with localized pain, an injection with a needle directly on the area to be treated substantially reduced pain after 30 minutes and completely reduced pain after 2 hours, with a lasting effect.

### Example 3.1—Study on Explants of Human Skin

### Principle of the Study

For the present study, an explant of frozen human skin was cut into disks with a diameter if 10⁻² m (1 cm).

5 series of explant samples to be tested—in triplicate—were prepared:
series 1: control samples (disks),
series 2: samples (disks) treated for 30 minutes;
series 3: samples (disks) treated for 60 minutes;
series 4: samples (disks) treated for 120 minutes;
series 5: samples (disks) treated for 240 minutes.

The treatment of the disks of the series 2-5 consisted in applying to each explant, 500 mg of the formula of Table 10 below.

TABLE 10

| Phase | Commercial name | % | INCI |
|---|---|---|---|
| B | Water | Q.S. | Aqua |
| A | Frametime CXG | 4.5 | Bentonite & Xanthan gum & |
| | marketed by Ephyla | | sodium stearoyl glutamate & |
| | | | citric acid |
| A | OLO3D marketed by | 4.0 | Stearalkonium Bentonite |
| | Ephyla | | |
| B | Concentrated extract | 30.0 | Anarcardium occidentale L |
| | (73%) of shells of | | (Cashew shell) extract |
| | cashew [nuts] obtained | | |
| | in Example 1.1 | | |
| Total | | 100 | |

The present formula was prepared as follows.

On the one hand, in order to obtain a homogenized mixture, the constituents of phases A were mixed at 20° C. with a mechanical mixer with blades or a rotor stator tool at a speed comprised between 3500 and 4000 rpm, for 7 minutes. On the one hand, the constituents of phases B were mixed at 20° C. with a mechanical mixer with blades or a rotor stator tool at a speed comprised between 3500 and 4000 rpm, for 7 minutes.

Finally, in order to obtain a homogenized composition, phase B was fed into phase A and the phases were mixed with a mechanical mixer with blades or a rotor stator tool at a speed of 4000 rpm for 5-7 minutes.

The treatment of the disks of series 2-5 took place as follows: 500 mg of the formula shown in table 10 hereinabove were applied to each sample, a sample corresponding to an explant disk placed in survival condition on physiological medium (6-well culture plate).

After treatment for the time period indicated hereinabove (between 30 minutes for series 2 and 240 minutes for series 5), the samples were removed and rinsed with physiological saline (in order to remove the treatment from the surface of the skin). The samples were then returned to a survival condition on physiological medium (in a CO₂ oven); then frozen.

A histological examination of human skin disks was carried out by the immunohistology laboratory after an HES (hemalum [solution]-eosin-saffron) coloration. All dermal and epidermal structures were evaluated.

### Conclusions

The histological analysis revealed a progressive change of skin alterations over time.

No major changes were noted on the disks treated for 30 minutes, but the appearance of possible elastosis in the superficial dermis was noted on the disks treated for 60 minutes.

At 120 minutes, nuclear alterations were noted in the epidermis without any significant inflammation.

At 240 minutes, epidermal necrosis associated with a more marked inflammatory infiltration was observed.

Thereby, the tested formula showed an action on the dermis and the epidermis, the specific action on the epidermis from 120 minutes on, which was confirmed at 240 minutes by necrosis or abrasion of the epidermis

### Example 3.2—Effect of the Pure Extract

3 patients were treated with the pure extract prepared in Example 1.2 hereinabove: 1 patient with acrochordon (soft fibroma or pendular fibroma) on the neck, 1 patient with vascularized ruby angioma on the cheek, 1 patient with dysplastic nevus on scapula.

### Example 3.2 b—Treatments

### Equipment and Method

The observations were made with a dermatoscope apparatus such as the FIREFLY Digital DE300 (zoom ×60).

### Preparing the Patients

The preparation of the 3 patients to be treated was carried out in the same way as in Example 2.1 b—Treatments.

### Applying the Treatment

The treatment was applied in the same way as in Example 2.1 b—Treatments.

### Care for the Treated Zone

The treatment care was carried out in the same way as in Example 2.1 b—Treatments.

### Follow-Up and Documentation

1. Daily observation with the FIREFLY Digital DE300 (zoom ×60) dermatoscope for the first 4 days.
2. Then observation every 2-3 days until D+14.
   Observations on a Patient with Acrochordon (Soft Fibroma or Pendular Fibroma) on the Neck
D0: after 4 hours; the treated area turned brown by a cornification effect; the acrochordon had stiffened and detached; no particular sensation;
D1: No particular sensation; the treated area had cornified;
D2: crust forming on the treated area; the border was red; slight itching;
D3: ditto D2 with hyperemic effect of diffusion around the treated area; slight swelling around the border; slight itching;
D5: the treated area had changed into a hard, deep crust; the border was still red; no itching;
D7: the treated area had become an already dry crust; no itching;
D10: the crust was detaching over 30-35% of the surface thereof, in particular on the border thereof;
D14: the crust had detached; the skin was healthy without any apparent scar; the pigmentation and the acrochordon (soft fibroma or pendular fibroma) had disappeared;
   Observations on the Patient with Vascularized Ruby Angioma on the Cheek
D0: after 4 hours; the treated area turned brown by a cornification effect; the vascularization seemed inflamed with bleeding under coagulation; small tingling sensation;
D1: the treated area had cornified; the vascularization had coagulated and a crust was forming; no particular sensation;
D2: ditto D1 with confirmation of a crust being formed;
D3: ditto D2 with slight hyperemic effect of diffusion around the treated area and of the crust formed; small itching;
D5: the treated area had changed into a crust; the border was red; small itching;
D7: the treated area had become a crust that was drying out; no particular sensation;
D10: the crust was dry and was significantly detaching around the border thereof;
D14: the crust had detached; the skin was healthy with no apparent scar; the pigmentation and the ruby angioma had disappeared.
   Observations on the Patient with Dysplastic Nevus on a Scapula
D0: after 4 hours; the pigmentation of the dysplastic nevus was reduced to a cluster; the vascularization appeared inflamed throughout the nevus area; sensation of tingling;
D1: the treated area had cornified, the traces of pigmentation of the nevus had completely disappeared; the vascularization had coagulated and stiffened; no particular sensation;
D2: ditto D1; slight hyperemic effect of diffusion around the treated area; slight sensation of itching;
D3: ditto D2 with hyperemic effect of diffusion around the treated area; said area was appreciably swollen; slight sensation of itching;
D5: the treated area had developed into a crust; the border showed red hyperemia; sensation of itching;
D7: the treated area had become a crust that was drying; the border showed red hyperemia as in D5; very slight sensation of itching;
D10: the crust was dry and seemed to detach over the border thereof; the border was showing hyperemia; no particular sensation;
D14: the crust had detached; the skin was healthy without any apparent scar; the dysplastic nevus had completely disappeared; hyperemia on the border was still present; no particular sensation.

The above was thus a coagulation leading to exfoliation; the treatment was particularly effective for removing, by necrosis and exfoliation, acrochordons (soft fibromas or pendular fibromas), scars, stretch marks, pigmentary lesions (melasma), neva, especially dysplastic neva, age spots or lentigos, unattractive moles such as ruby angiomas neva. The coagulation of blood micro-vessels observed in the case of ruby angiomas further sustains applications to stop bleeding, especially when skin cuts occur and affect the dermis and/or hypodermis.

It should be noted that the content of the present composition penetrates through the epidermis to reach the dermis as well, which makes it possible, in other volunteers, at the dermal-epidermal junction, to inhibit pain by cell necrosis of cells inflamed at the nerve endings, said composition thus being particularly recommended for the curettage of wounds, in which case the application should not exceed 3 hours and [should be applied for] at least 1 hour: the application as a poultice acts, without any mechanical action, as a curettage.

The pure extract prepared in Example 1.2 hereinabove was studied by immunofluorescence with P53 labeling and Caspase-3

Immunofluorescence with P53 Labeling

The quantitative analysis of the marker of P53 which is the epidermal differentiation and repair protein was performed according to the protocol below.

Table 11 shows the differential expression of P53 after treatment with the pure extract.

TABLE 11

| | | Treated for | |
|---|---|---|---|
| Compartment | Control | 240 minutes | Variation (%) |
| Epidermis | 58.00 | 62.33 | +7.46% |
| Dermis | 55.76 | 55.82 | +0.11% |

The significant increase in P53 in the epidermis (+7.46%) vs the quasi-total stability in the dermis (+0.11%) demonstrates that the pure ethanolic extract exerts an action which is selective and differentiating on epidermal keratinocytes.

The significant increase in P53 in the epidermis confirms that the pure extract specifically induces a controlled epidermal turnover process, characteristic of a differentiated exfoliation.

Immunofluorescence with Caspase-3 Labeling

Quantitative analysis of the Caspase-3 marker which is a marker of apoptosis, was performed according to the protocol below.

### Protocol

Table 12 shows the expression of Caspase-3 after treatment with the pure extract.

TABLE 12

| | | Treated for | |
|---|---|---|---|
| Compartment | Control | 240 minutes | Variation (%) |
| Epidermis | 51.59 | 52.55 | +1.86% |
| Dermis | 52.58 | 54.63 | +3.90% |

### Results

The moderate increase of Caspase-3 in the epidermis (+1.86%) confirmed the involvement of keratinocytes in a controlled apoptotic process consistent with the exfoliation mechanism observed histologically.

The pharmaceutical compositions of the present invention offer a number of significant benefits over prior art approaches to skin exfoliation and dermal coagulation, each of which is supported by the experimental data and clinical observations described herein.

The compositions of the present invention act primarily via an intracellular route and preferentially target deviant, abnormal, or otherwise compromised cells, such as those constituting warts, nevi, angiomas, acrochordons, keratoses, and cellulitic adipocytes, while sparing surrounding healthy tissue to the extent possible. This selective mechanism is supported by the immunofluorescence data of Example 3.2, which demonstrates a significant and specific increase in the epidermal differentiation and repair marker P53 in the epidermis (+7.46%) compared to quasi-total stability in the dermis (+0.11%) following treatment, indicating a controlled and differentiated action on targeted keratinocytes rather than indiscriminate tissue destruction. Furthermore, the clinical observations across Examples 2.1b, 2.2b, 2.3b, and 3.2b consistently show complete resolution of treated lesions—including plantar warts, fatty cysts, acrochordons, ruby angiomas, and dysplastic nevi—with healthy, scar-free skin at Day 14, confirming that the surrounding tissue is not permanently damaged by the treatment.

Unlike conventional chemical exfoliants, which act primarily at the level of the epidermis or superficial dermis, the compositions of the present invention are capable of penetrating to the dermis and even the hypodermis following superficial topical application. This is demonstrated by the histological analysis of Example 3.1, which shows progressive alterations from the superficial dermis at 60 minutes to full epidermal necrosis with inflammatory infiltration at 240 minutes, and by the clinical resolution of hypodermic fatty cysts (Example 2.1b) and deep plantar warts (Examples 2.1b and 2.9) by topical and injectable application, respectively. This depth of action is particularly advantageous because it allows treatment of conditions located in the deep dermis and hypodermis that are not accessible to conventional topical chemical exfoliants, without the need for invasive physical procedures such as laser, electrical, thermal, or cryogenic dermal coagulation.

The physical methods of dermal coagulation known in the prior art, including laser-induced, thermal, electrical, and cryogenic coagulation, generally result in non-selective cell destruction and are frequently painful or stressful for the patient. Phenol-based chemical exfoliants, while effective, present risks of skin sensitization and irritation. By contrast, the compositions of the present invention are stable, non-irritating, non-toxic, and non-allergenic, as demonstrated by the absence of pain, allergic reaction, or systemic toxicity in all volunteer patients across Examples 2.1b, 2.2b, 2.3b, and 3.2b. Specifically, the clinical observations consistently record no particular sensation or pain during or following application in the great majority of treated patients, with only mild and transient itching or tingling reported in a small number of cases during the crust formation phase, which resolved without intervention. No scarring was observed in any treated patient at the end of the follow-up period.

The present invention is not limited to any single formulation type or route of administration, which provides significant clinical flexibility. As demonstrated in the working examples, the cell-based coagulation activity of anacardic acid is effective whether delivered as a substantially oily topical composition that penetrates to the hypodermis (Example 2.1a), as an O/W Pickering emulsion that provides controlled, gradual release of anacardic acid to the epidermis through encapsulation of the oily phase in microcapsules of approximately 5 to 10 micrometers in diameter (Examples 2.2a, 2.3a), as a transdermal patch composition applied over a period of 2 to 6 hours for targeted deep coagulation (Examples 2.5, 2.6), or as a sterilized injectable composition for the treatment of deep lesions, subcutaneous hemorrhage, and localized pain (Examples 2.7-2.9). This versatility allows the clinician to select the most appropriate formulation depending on the depth, nature, and location of the tissue to be treated.

Beyond its primary use as a cell-based coagulant for skin exfoliation, anacardic acid has been found to exhibit several additional therapeutic benefits that are mechanistically consistent with its cell-based coagulation activity. First, the coagulation of blood microvessels observed during treatment of ruby angiomas and superficial vascular lesions (Examples 2.2b, 2.3b, 3.2b) supports the use of the compositions of the present invention to stop bleeding in skin cuts affecting the dermis and/or hypodermis. Second, the compositions have been found to inhibit localized pain through necrosis of inflamed cells at nerve endings, with pain substantially reduced within 30 minutes and completely eliminated within 2 hours following injectable administration (Examples 2.7-2.9), and with comparable effects observed via topical application in wound curettage settings (Examples 2.2a, 2.3a). Third, the compositions have demonstrated the ability to reduce and eliminate subcutaneous fatty structures, including cellulite and fatty cysts, through coagulation of adipocytes (Example 2.1b), providing an anti-cellulite and lipoma-reduction benefit. Each of these benefits arises directly from the cell-based coagulation mechanism of anacardic acid as defined and demonstrated herein.

The articles “a” and “an” may be employed in connection with various elements and components of compositions, processes described herein. This is merely for convenience and to give a general sense of the compositions and processes. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description is by way of example only.

1. A pharmaceutical composition comprising anacardic acid for use as a cell-based coagulant.

2. The pharmaceutical composition according to claim 1 wherein the anacardic acid is selected from 6-n-pentadecyl salicylic acid, 6-[8(Z)-n-pentadecenyl] salicylic acid, 6-[8(Z), 11(Z)-n-pentadecadienyl] salicylic acid, 6-[8(Z), 11(Z), 14(Z)-n-pentadecatrienyl] salicylic and mixtures thereof in all proportions.

3. The pharmaceutical composition according to claim 1, wherein the anacardic acid is selected from 6-n-pentadecyl salicylic acid, 6-[8(Z)-n-pentadecenyl] salicylic acid, as well as mixtures thereof in all proportions.

4. The pharmaceutical composition according to claim 1, further comprising an extract of shells of cashew nut or western Anacardium L., said extract containing anacardic acid.

5. The pharmaceutical composition according to claim 1, comprising from 0.1% to 100.0% by weight of anacardic acid relative to a total weight of the composition.

6. The pharmaceutical composition according to claim 1, containing from 0.1 to 99.9% by weight, relative to a total weight of the composition, of an oily phase comprising at least one oil chosen from vegetable oils, squalane, squalene and esters of vegetable fatty substances or obtained by biotechnology.

7. The pharmaceutical composition according to claim 6, wherein the vegetable oily phase comprises at least one vegetable oil chosen from the group formed by sunflower oil, rapeseed oil, olive oil, camelina oil, peanut oil, coconut oil, grape seed oil, castor oil, argan oil, Djansang oil, desert date oil, hemp oil, Nigella oil, Barbary fig oil, macadamia oil, soybean oil, palm and palm tree oil, Tamanu oil, Kukui nut oil (or candle nut), sesame oil, linseed oil, walnut oil, hazelnut oil, almond oil, baobab oil, passion fruit oil, Brazil nut oil, hibiscus oil, pumpkin seed oil, Luffa oil, Carapa oil, evening primrose oil, borage seed oil, avocado oil, sea buckthorn oil, apricot kernel oil, cherry kernel oil, apple seed oil, pomegranate oil, jojoba oil, rosehip seed oil, plum kernel oil, shea butter, cocoa butter, Kokum butter, mango butter, Moabi butter, Karanja butter, Tucuma butter, Cupuaçu butter, Buriti butter, Murumuru butter, Kombo butter, Kpangnan butter and triglycerides, in particular of caprylic/capric acids.

8. The pharmaceutical composition according to claim 7, wherein the vegetable oily phase comprises caprylic/capric acids.

9. The pharmaceutical composition according to claim 1, wherein said composition comprises an O/W Pickering emulsion containing an aqueous phase, a vegetable oil phase and an organically modified phyllosilicate.

10. The pharmaceutical composition according to claim 9, wherein the organically modified phyllosilicate is present in an amount ranging from 2% to 20% by weight relative to a total weight of the composition.

11. The pharmaceutical composition according to claim 10, wherein the organically modified phyllosilicate is present in an amount ranging from 2.5% to 10% by weight relative to the total weight of the composition.

12. The pharmaceutical composition according to claim 11, wherein the organically modified phyllosilicate is present in an amount ranging from 3.5% to 8% by weight relative to the total weight of the composition.

13. The pharmaceutical composition according to claim 1, comprising 0.001 to 20% by weight of at least one cosmetic agent chosen from humectants, thickeners, texturing agents, emulsifiers, dispersants, foaming agents, emollients, preservatives, colorants, plant extracts, plant fibers, minerals, pH adjusting agents, active ingredients and perfumes.

14. The pharmaceutical composition according to claim 1, wherein the composition is present in an amount sufficient to treat adipocytes and impart a slimming and/or anti-cellulite effect to the skin, and/or lead to a reduction of lipomas.

15. The pharmaceutical composition according to claim 1, wherein the composition is present in an amount sufficient to treat an inflammatory cellular environment around nerve cells and act as a painkiller agent.

16. The pharmaceutical composition according to claim 1, wherein the composition is present in an amount sufficient to reduce fibrosis or cysts.

17. The pharmaceutical composition according to claim 1, wherein the composition is present in an amount sufficient to exfoliate the skin and/or surface flaws.

18. The pharmaceutical composition according to claim 17, wherein the surface flaws are lentigos, acrochordons, cutaneous cysts, molluscums, keratoses and xanthelasmas.

19. The pharmaceutical composition according to claim 17, wherein the composition is present in an amount sufficient to provide at least one treatment selected from the group consisting of:
a treatment of atrophic scars, a curettage and a treatment of warts, a treatment of herpes pimples, a treatment of acrochordons, a treatment of pigment spots or lentigos, a treatment of keratoses, a treatment of xanthelasmas, the curettage of wounds, a treatment of telangiectasia, a treatment of varicosities, a treatment of angiomas, a treatment of ruby spots, a treatment of superficial blood vessels of the skin.
